(19) United States
(12) Patent Application Publication
(10) Pub. No.: US 2007/0290065 A1
(43) Pub. Date: Dec. 20, 2007

(54) Volatile Material Dispensing Apparatus

(75) Inventors: Colin Brown, Bracknell Berkshire (GB); Guy Edward Naish, Bicester Oxfordshire (GB); Kishen Gohil, New Malden Surrey (GB); Simone Anderson, Ashford Middlesex (GB)

Correspondence Address: NORRIS, MCLAUGHLIN & MARCUS, 875 THIRD AVE, 18TH FLOOR, NEW YORK, NY, 10022, US

(73) Assignee: Givaudan SA, Vernier (GB)

(21) Appl. No.: 11/574,545

(22) PCT Filed: Sep. 2, 2005
(86) PCT No.: PCT/CH05/00517

§ 371 Date: May 14, 2007

(30) Foreign Application Priority Data
Sep. 1, 2004 (JP) 2004-254650

(51) Int. Cl. A61L 9/04

(52) U.S. Cl. 239/44

(57) ABSTRACT

A device adapted to disseminate a volatile liquid, such as a fragrance, into an atmosphere comprises a reservoir (1) containing the liquid (6) and a capillary means (8) for conveying the liquid from the reservoir to the atmosphere. The reservoir has an upper orifice (2) into which the capillary means fits loosely. This orifice is in liquid-tight contact with a tubular member (3), which extends downwards into the reservoir, the tubular member and reservoir being proportioned such as to define a liquid-retaining volume regardless of the orientation of the device. The liquid is preferably introduced by means of a sealed cartridge (5), which may be supplied separately or as an integral part of the device. The device allows the use of a wide variety of capillary means and has the advantage of being spillproof.

This invention relates to devices for dispensing volatile materials into an atmosphere.

Devices for dispensing volatile materials into an atmosphere, often (but not always) an interior atmosphere such as that in a room or conference venue, are well known. The volatile materials include fragrances, insecticides, medicaments and fungicides. A typical device comprises a reservoir for holding the liquid and a means of conveying it from the reservoir to the atmosphere and there causing it to evaporate. One of the most common conveying/evaporating means is a wick made of a porous material that conveys the liquid by capillary action. This wick is generally fitted to the reservoir in a liquid- and vapour-tight fashion, so that liquid can only escape by capillary action through the wick. To allow for pressure equalization and to permit excess liquid to drain back into the reservoir, the means sealing the wick in place (generally an insert or cap) comprises a passage of small cross-sectional area, this allowing communication between atmosphere and reservoir interior, such that liquid can continue to evaporate and, if necessary, that excess liquid on the wick can drain back into the reservoir.

This kind of device has two problems. First of all, only the wick sealed into the reservoir can be used—there is no possibility of using another type of wick, or multiple wicks. Secondly, if the device is inadvertently knocked over, liquid can leak from the pressure equalization passage. Alternative wicks can be desirable, for both aesthetic and practical reasons. For example, it is desirable to use dried natural materials, such as plant stems, as wicks. These can only be installed in open-necked vessels, which are even more prone to spillage when knocked over than are devices with sealed wicks.

It has now been found that these problems in the art can be substantially and even completely overcome by the use of a particular device. The invention therefore provides a device adapted to disseminate a volatile liquid into an atmosphere, the device comprising a reservoir containing the liquid and a liquid-transporting capillary means for conveying the liquid from the reservoir to the atmosphere, the reservoir having an upper orifice into which the capillary means fits loosely, and, fixed to the reservoir in liquid-tight contact and extending downwardly from the orifice, a tubular member, the tubular member and reservoir being proportioned such as to define a liquid-retaining volume regardless of the orientation of the device.

By “loosely” is meant that the capillary means is not tightly sealed in the reservoir such that here is no free space around it. Typically, the orifice is substantially larger in diameter than the capillary means (or the combined diameters of a number of capillary means, should a number e used). The capillary means can be freely inserted in the reservoir, and if necessary freely withdrawn therefrom. It is a feature of this invention that the orifice is partially open to the atmosphere, but that accidental spillage cannot take place.

The reservoir is a vessel with an upper orifice and may be made of any desirable or appropriate material, such as plastics, glass, metal or ceramic. The precise configuration of the reservoir shall be hereinafter described, in conjunction with the other elements of the device, but a wide variation of shapes is possible from the purely functional to the highly aesthetic. The variety is much wider than that possible by using known technology.

The capillary means may be any means that is capable, by capillary action, of transporting a volatile liquid from the reservoir and permitting it to evaporate into the atmosphere. It may be, for example, a wick of the type well known to and widely used by the art. Alternatively, it may be a rod with external capillaries formed therein as described, for example, in U.S. Pat. No. 4,913,350. A further alternative is the combination of wick and capillary sheet as described in International Patent Application PCT/CH04/000102. A further alternative is dried plant material, such as plant stems. This has the advantage of being cheap, environmentally friendly and aesthetically pleasing. The advantage of this invention is that, because the capillary means may be loosely inserted into the reservoir, any kind of known capillary means may be used, or even two or more different ones at the same time, depending on the aesthetic or practical effect desired, again a versatility not possible in the known art.

The reservoir has an upper orifice for insertion of the capillary means and for adding the liquid. Depending downwards into the reservoir from this orifice is a tubular member, that is, a member that extends completely around the boundary of the orifice. The tubular member may be provided by any convenient means. For example, it may be a separate component, inserted into the reservoir and held in place by any convenient means, for example, by adhesive, screw threads or a snap fitting. In this case, the join, where tubular member and reservoir meet, must be liquid-tight. Preferably the tubular is formed integrally with the reservoir, such that the tubular member is a re-entrant portion of the reservoir. Such a combined reservoir and tubular member may be easily made by methods well known to the art, such as casting or moulding. In this case, there is no problem with attaining liquid tightness. The tubular member may be a downwards extension of the boundary of the orifice itself, that is, the transverse cross-section of the tubular member matches the shape of the orifice. Alternatively, it may be a different shape. It may have parallel sides, or it may taper or widen as it moved away from the orifice.

The relationship between the reservoir and the tubular member should be such there is defined a liquid-retaining volume when the device is in any orientation, even if completely inverted. This ensures that, should the reservoir be accidentally knocked over, little or no liquid will be spilled. The basic principle here is that the liquid level should never reach the lower end of the tubular member. The two factors that have to be regulated to ensure that this happens are:
the volume of the reservoir;
the distance into the reservoir which the tubular member extends.

At this point, it is worth mentioning that the volume of the liquid present also has a bearing on whether there will be spillage should the reservoir be accidentally inverted, but it is self-evident that the liquid level should not be higher than the bottom of the tubular member, otherwise the risk of liquid running out if the reservoir is tipped over is very great.

A similar principle was utilized in European Patent 1 088 562, in which the use of a long sleeve tightly fitted around a wick in a reservoir of sufficient volume ensures that the wick is removed from the liquid in the reservoir when the reservoir is tilted or even inverted. This is used in conjunction with a means of utilizing a variety of liquids in individual reservoirs in a common apparatus. However, there is no indication in this document (which utilizes tightly-fitting wicks exclusively) that it is possible or desirable to make an open-ended, spill-proof reservoir with loosely-fitting capillary means.

The interrelationship between volume of reservoir and extension of tubular member into the reservoir will differ, depending on the volumes and shapes of reservoir and tubular member, which in turn will be regulated by practical and aesthetic demands, but the skilled person, having this concept of a reservoir and tubular member that will essentially not spill any liquid, even if completely inverted, will be able to provide the necessary interrelationship as part of the normal skill of the art.

The liquid may be introduced by simply pouring it through the orifice into the reservoir. If the orifice is small, this can be a problem and lead to splashing or spillage. However, a novel construction avoids these problems completely. The invention therefore also provides a device adapted to disseminate a volatile liquid into an atmosphere, the device comprising a reservoir containing the liquid and a liquid-transporting capillary means for conveying the liquid from the reservoir to the atmosphere, the reservoir having an upper orifice into which the capillary means fits loosely, and, extending downwardly from the orifice, a tubular member, the tubular member and reservoir being proportioned such as to define a liquid-retaining volume when the device is in an inverted position, the liquid being supplied in a sealed cartridge that is openable when the cartridge is in place in the reservoir.

The cartridge may be any suitable cartridge capable of holding a liquid. It should be capable of holding an appropriate amount of liquid, to comply with the desire of non-spillage, should the reservoir be tilted or inverted. It is preferably in the form of an elongate cylinder. It is naturally of such cross-section that the desired capillary means can also be inserted into the reservoir.

This embodiment can take many different forms. For example, the cartridge may be supplied separately from the reservoir and the reservoir provided with a structure adapted to open the cartridge upon insertion into the reservoir. One way of achieving this is to seal the lower end of the cartridge with a foil of metal or plastics, which foil is adapted to be penetrated by an opening element associated with the reservoir. This opening element is typically a prong extending from the floor of the reservoir, and disposed such that inserting the cartridge and pushing it home opens the cartridge and releases the liquid.

In a further variant of this embodiment, a liquid-filled cartridge has an openable end that is affixed to the floor of the reservoir, such that the action of moving the cartridge away from the floor causes the cartridge to open and liquid to come out. Again, this may be done by closing the cartridge with a foil, which is then adhered to the floor of the reservoir. Alternatively, the foil may be replaced by a screw cap or a protrusion of the base that acts as a stopper for the cartridge. There are a number of possible variants of this embodiment that lie within the skill of the art and all are encompassed by it.

The capillary means may be separate from the cartridge and inserted loosely. However, in the case of a cartridge that is separate from the reservoir, the capillary means may be attached to the cartridge and may be supplied and inserted with it. This can be achieved by any suitable means, but preferably the capillary means is affixed by any suitable means around the circumference of the cartridge and the orifice of the reservoir is sufficiently wide to permit the combined cartridge and capillary means to be inserted. The capillary means should be place such that their bottoms will be immersed in the liquid when it is released, but the skilled person can readily provide this.

The device according to the invention is considerably more versatile than known devices based on capillary mechanisms. A wider variety of forms is available, adding to the aesthetic appeal, and the nature of the capillary means may be selected from the wide range available to the art, as opposed to the normal restriction to one type. The device and its components may be easily and cheaply manufactured from known materials.

The most important attribute of the device of the present invention is the fact that spills of liquid may be completely avoided when the device is accidentally tipped over. The invention therefore also provides a method of preventing spillage of volatile liquid from a device adopted to disseminate the liquid into an atmosphere, the device comprising a reservoir containing the liquid and liquid-transporting capillary means extending between the liquid and the atmosphere, the reservoir being open to the atmosphere via an orifice at the top thereof, the method comprising the provision in the reservoir of a tubular member extending downwards into the reservoir from the orifice from a liquid-tight contact with the top of the reservoir, the length of the tubular member and the volume of the reservoir being so proportioned that there is provided sufficient volume to contain the liquid in any orientation of the device.

An additional advantage of the present invention is that it allows easier compliance with the legal requirements of many countries. These define a maximum amount of headspace permissible in a liquid-filled container, and it is generally so small as to require a high liquid level. This means that the liquid level will be so high that it is practically impossible to provide a device for a liquid level sufficiently low to permit the principle of the present invention to work. In the case of the present invention, especially the cartridge variant, there is no such problem.

The invention will now be further described with reference to the accompanying drawings, which depict a preferred embodiment, and which are not intended to be limiting in any way.

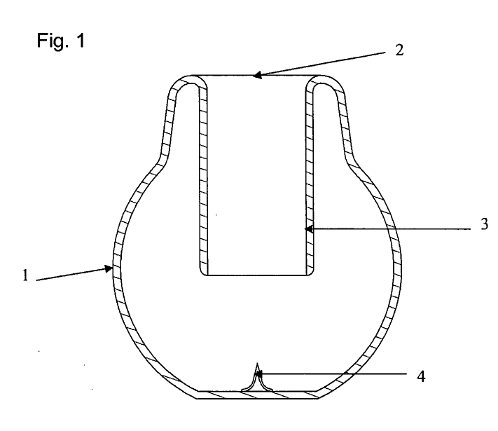

FIG. 1 depicts a longitudinal cross-section through a reservoir according to the invention.

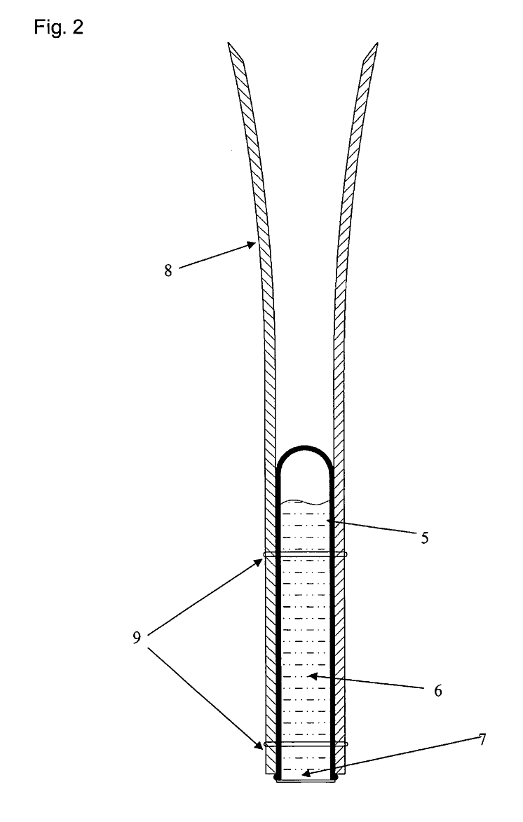

FIG. 2 depicts a longitudinal cross-section through a liquid-carrying cartridge equipped with capillary means and adapted to fit into the reservoir of FIG. 1.

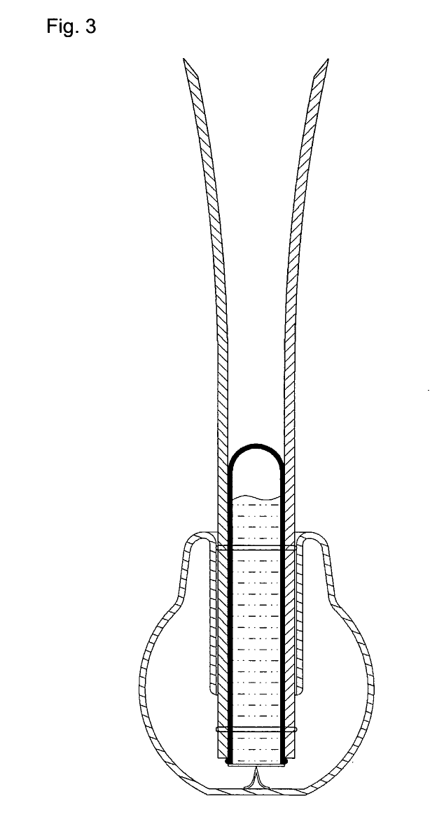

FIG. 3 depicts a complete device, with the cartridge of FIG. 2 inserted into the reservoir of FIG. 1.

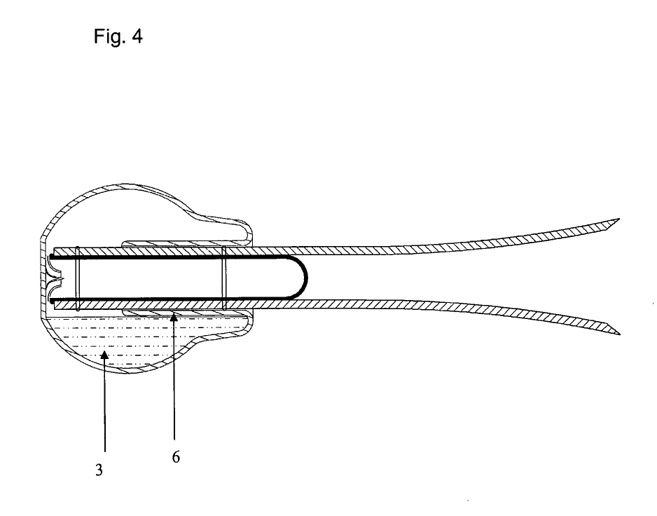

FIG. 4 depicts the device of FIG. 3 tipped on its side, to show the effect of the invention.

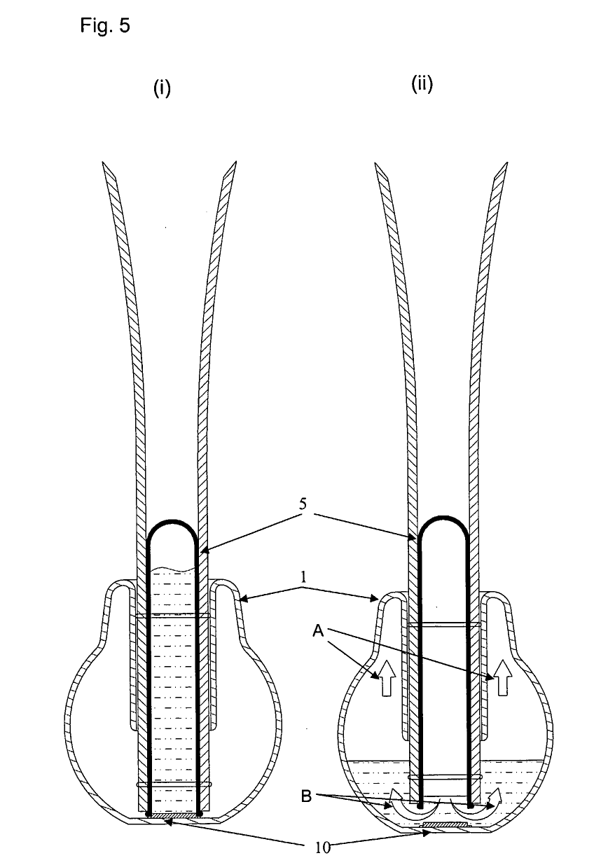

FIG. 5 depicts a further embodiment of the invention.

In the embodiment of FIG. 1, a reservoir 1 is a vessel that is bulbous at the lower end that sits on a support surface (to make the device more stable. At its upper end, it has an orifice 2, adapted to receive capillary means and to permit refilling. The orifice 2 merges into a tube 3 that extends downwards into the reservoir. Rising upwards from the bottom of the reservoir and positioned directly under the tube 3 is a projection 4. This projection is adapted to pierce the bottom of a liquid-containing cartridge inserted into the reservoir.

FIG. 2 shows a cartridge suitable for use with the reservoir of FIG. 1 and FIG. 3 shows it in place, prior to its being punctured, with resultant release of the liquid. The cartridge 5 has a form similar to a classical test-tube. It is filled with liquid 6 and is mounted with its mouth downwards, the open end being sealed by a tearable metal foil barrier 7. To the sides of the cartridge are attached capillary means 8 (in this case dried plant stalks), attachment being made by plastic ties 9. As shown in FIG. 3, the cartridge and associated capillary means are insertable into the orifice 2 of the reservoir 1. When this is done and the cartridge is pushed home, the projection 4 breaks through the foil barrier 7 and releases the liquid in the reservoir 1.

In FIG. 4, the device of FIG. 3 has been tipped on its side. As can be seen, the level of the liquid 6 does not reach the bottom of the tube 3, so there is no leakage, even if the vessel were to be fully inverted.

FIG. 5 shows two views of a further embodiment. In this embodiment, a tearable foil 7, which closes a cartridge 5 and therefore holds liquid in the cartridge, is affixed to the bottom of the reservoir 1, as can be seen in FIG. 5(i). The device is thus supplied as a single unit. The liquid is released by pulling the cartridge 5 in the direction of the arrows A as shown in FIG. 5(ii). This will cause the cartridge to separate from the foil, causing liquid to flow into the reservoir

1. A device adapted to disseminate a volatile liquid into an atmosphere, the device comprises
a reservoir containing the liquid,
a liquid-transporting capillary means for conveying the liquid from the reservoir to the atmosphere,
the reservoir having an upper orifice into which the capillary means fits loosely, and,
a tubular member affixed to the reservoir in liquid-tight contact and extending downwardly from the orifice, the tubular member and reservoir being proportioned such as to define a liquid-retaining volume regardless of the orientation of the device.

2. The device according to claim 1, wherein the liquid is supplied in a sealed cartridge that is openable when the cartridge is in place in the reservoir.

3. The device according to claim 2, wherein the cartridge is supplied separately from the reservoir and the reservoir provided with a structure adapted to open the cartridge upon insertion into the reservoir.

4. The A device according to claim 3, wherein the cartridge is sealed with a tearable foil and the structure is a prong mounted on a floor of the reservoir, such that the prong penetrates the foil when the cartridge is inserted into the reservoir.

5. The A device according to claim 2, wherein a liquid-filled cartridge is equipped with an openable end (10) that is affixed to a floor of the reservoir, such that the action of moving the cartridge away from the floor causes the cartridge to open and liquid to come out.

6. (canceled)

7. A method of preventing spillage of volatile liquid from a device adopted to disseminate the liquid into an atmosphere, the method comprising the steps of
providing a reservoir containing the liquid;
providing liquid-transporting capillary means (8) extending between the liquid and the atmosphere, the reservoir being open to the atmosphere via an orifice (2) at the top thereof,
providing a tubular member (3) downwardly into the reservoir from the orifice, and
proportioning a length of the tubular member and a volume of the reservoir such that there is provided sufficient volume to contain the liquid in any orientation of the device without spilling the liquid.
